(19) United States
(12) Patent Application Publication
(10) Pub. No.: US 2025/0361564 A1
(43) Pub. Date: Nov. 27, 2025

(54) ORTHOGONAL VALIDATION OF TUMOR ASSAYS

(71) Applicant: SAGA Dx, Inc., Morrisville, NC (US)

(72) Inventors: Miguel Alcaide, Lund (SE); Anthony Miles George, Lund (SE); Lucia Oton, Lund (SE); Yilun Chen, Lund (SE)

(21) Appl. No.: 19/213,729

(22) Filed: May 20, 2025

Related U.S. Application Data
(60) Provisional application No. 63/650,048, filed on May 21, 2024.

(51) Int. Cl. C12Q 1/6886; C12Q 1/6806; C12Q 1/6874

(52) U.S. Cl. CPC C12Q 1/6886; C12Q 1/6806; C12Q 1/6874; C12Q 2600/156

(57) ABSTRACT

The invention provides methods for analyzing tumor nucleic acid from a tumor from a subject to discover one or more variants that are specific to the tumor and confirming by orthogonal testing that nucleic acid of the tumor harbors the variants and that the variants are specific to the tumor and thus useful as a tumor biomarker in an independent assay for the presence of the tumor in the subject.

### TECHNICAL FIELD

The invention relates to an orthogonal validation of tumor assays.

### BACKGROUND

There are roughly 1.9 million new cancer cases diagnosed every year in the United States alone. Various forms of cancer have been shown to attack nearly every part of the human body. Cancer often requires aggressive and expensive treatment. Despite rigorous treatment, cancer can still recur in 15-75% of patients. Potential recurrence can be monitored in a multitude of ways including scans and bloodwork but, oftentimes, recurrent cancer is noticed too late, if at all. This can lead to great suffering for both the patient and their loved ones.

When a tumor is found in a patient, a doctor may perform a biopsy to take a sample of the tumor that can be analyzed or stored. The patient then may be treated to remove or eradicate the tumor. Tumor treatment may involve surgical removal, immunotherapy, radiation therapy, or some other such approach. One clinically important question after treatment to remove a tumor is whether the removal was complete, or whether some small number of tumor cells may still be present with the future potential for the tumor to grow back. It is thought that molecular information from a sample could be used as a biomarker to guide a future test for evidence of the continuing presence of some small number of residual tumor cells. Such a test is sometimes referred to as a test for minimal residual disease (MRD). One approach to a test for MRD would be to look for circulating tumor DNA (ctDNA) harboring sequences that are found uniquely in the tumor biopsy. However, due to a variety of factors, such as very low amount of ctDNA in a blood draw, biases in PCR, and difficulties with homopolymer runs and short read assembly in next-generation sequencing (NGS), some tests for MRD may be prone to false negatives and/or false positives.

### SUMMARY

The present invention provides improved methods for the detection of tumors and evidence of MRD. In methods of the disclosure, tumor nucleic acid is sequenced, and the sequence data are analyzed to identify one or more tumor-specific variants that constitute a tumor mutation profile or tumor signature. Each variant is found specifically in tumor nucleic acid and not in healthy, non-tumor nucleic acid from the subject. In that sense, the variant is specific to the tumor and is referred to as a tumor variant. Once a tumor variant is identified by sequencing the tumor nucleic acid and analyzing the sequence data, the tumor variant is validated, as a true tumor variant, by an orthogonal detection method that uses different molecular or biochemical techniques to detect or confirm variants. In the event that the primary, sequencing-based detection technique is associated with any bias from amplification, systemic error associated with short-read assembly, or struggles with homopolymer run, the orthogonal technique had different biases or systematic errors. Because the detected tumor variant is validated by an orthogonal detection technique with biases or errors that are unlike those of the primary detection technique, the tumor variant is understood to be truly a tumor-specific variant, that will be found in the tumor genome but not found in non-tumor DNA from the same subject.

In certain embodiments, the invention provides methods to create a tumor signature or tumor mutation profile in which structural variants (SVs) specific to a tumor are identified. Methods include sequencing tumor nucleic acid, from a tumor from a subject, to obtain sequence data and analyzing the sequence data to identify a tumor variant, present in the tumor nucleic acid but not present in healthy, non-tumor nucleic acid from the subject. The identified tumor variant is then validated by performing a second detection technique to obtain results that are consistent with the presence of the tumor variant in the tumor nucleic acid. The second detection technique is orthogonal to the sequencing step at least in that sources of bias or error associated with the sequencing technique are not implicated in the orthogonal detection technique.

In some embodiments, sequencing the tumor nucleic acid includes obtaining the tumor nucleic acid from a sample such as a formalin-fixed, paraffin embedded (FFPE) slice of the tumor. A sequencing library may be prepared (by nucleic acid fragmentation, end-repair, adaptor ligation, and amplification, for example). The sequencing may be next-generation sequencing. In particular, the sequencing maybe low-pass, whole genome sequencing (LP-WGS). Sequencing may generate a plurality of short sequence reads that may be assembled and/or mapped to a reference to identify an SV specific to the tumor. The reference may be a genome sequence obtained from “matched normal” sequence reads obtained by sequencing DNA from non-tumor cells from subject. The tumor variant may be identified computationally and even without using a matched normal reference. The tumor variant may be identified, in another example, by mapping paired end reads to a published human genome reference and identifying read pairs that map to the reference in a pattern that is discordant with an insert size of the paired end reads.

Once the tumor variant has been identified by the primary identification technique such as NGS (e.g., optionally by LP-WGS on an NGS platform), the tumor variant is validated by an orthogonal detection technique. Suitable orthogonal detection techniques include, for example, digital PCR, Sanger sequencing, atomic force microscopy (AFM), restriction fragment length polymorphism (RFLP), amplified fragment length polymorphism (AFLP), optical mapping, fluorescent in situ hybridization (FISH), DNA microarray, in vitro transcription (IVT), protein expression or detection, enzyme-linked immunosorbent assay (ELISA) mass spectrometry, true single molecule sequencing, others, or any combination thereof.

When the orthogonal validation technique finds and confirms the tumor variant, the tumor variant may be taken to be a true, tumor-specific variant and may be used subsequently as a biomarker in an assay for evidence of the presence of the tumor in the subject.

Various features and options are within the scope of the disclosure. Multiple biopsy methods may be used here including wherein a first and a second tumor sample are taken or wherein the first and second tumor samples are derived from the same sample. The present invention may also be used to identify (and validate) variants that are not considered to be SVs such as a single nucleotide polymorphism (SNP), an indel (insertion-deletion), a single nucleotide variant (SNV), a deletion, a rearrangement, an amplification, or the like. The tumor mutation, in many cases, is greater than 10 bp in length. The present invention also allows for a high degree of variability in orthogonal testing of the patient sample. The patient sample may blood but may also be saliva, plasma, urine, CSF, stool, tumor biopsy, lymphatic fluid, or the like. This sample may be any in which ctDNA may be found. The present invention also allows for different types of orthogonal testing such as digital PCR (dPCR), Quantitative PCR (qPCR), ELISA, nanopore, NGS, FRET-seq, or any other next generation sequencing (NGS) method. In certain embodiments of the present invention, the orthogonal testing may be biased by removing or not analyzing specific primers or variants based on results obtained from previous testing and/or sequencing. In certain embodiments, the tumor sample may be further tested to confirm the nature of the tumor variant (e.g., a third detection orthogonal to primary and first orthogonal). According to another embodiment, the detection of one or more tumor variant(s) constitutes a positive test.

### DETAILED DESCRIPTION

The invention provides methods for tumor analyzing nucleic acid from a tumor from a subject to discover one or more variants that are specific to the tumor and confirming by orthogonal testing that nucleic acid of the tumor harbors the variants and that the variants are specific to the tumor and thus useful as a tumor biomarker in an independent assay for the presence of the tumor in the subject. Variants that are found in the genome of the tumor and that are specific to the tumor and thus not also found in the genome of non-tumor cells from the subject may thus be referred to as tumor-specific variants or tumor variants. Methods of the invention are useful to discover tumor-specific variants and, due to the orthogonal validation, the tumor specific variants are detected and reported with minimal false positives or false negatives. Those tumor-specific variants may then subsequently be used in assay for minimal residual disease (MRD).

Methods may include obtaining tumor nucleic acid, e.g., from a tumor biopsy, sequencing the tumor nucleic acid to obtain sequence data, and analyzing the sequence data to detect tumor variants. In some embodiments, the sequence data are obtained by sequencing DNA from a formalin-fixed, paraffin embedded slice of the tumor. Methods of the disclosure may include extracting nucleic acid from a tumor biopsy or other fixed sample and preparing a sequencing library. In some embodiments, nucleic acid such as DNA is extracted from a formal-fixed, paraffin embedded (FFPE) tissue sample.

### FFPE DNA Extraction

Methods of the disclosure may include obtaining nucleic acid from a formalin-fixed, paraffin embedded slice of a tumor, so that the tumor nucleic acid may be sequenced. Tissue obtained by biopsy or surgery for pathological examination may be fixed in a fixative, such as formalin and embedded in paraffin, yielding formalin fixed, paraffin embedded (FFPE) blocks. Small (5 micrometer-thick) sections may be sliced from the blocks and stained for microscopic analysis. Such slides and the FFPE blocks are typically retained as a pathology archive.

Methods herein may use protocols for extracting DNA from FFPE samples and preparing high-quality sequencing libraries from the FFPE-extracted DNA. To extract nucleic acid, the sample is loaded into a tube such as 0.5 mL screw-cap microcentrifuge tube. A tissue lysis buffer and proteinase K (PK) solution mix may be added to the tube. Such materials may be obtained from a source such as Covaris (Woburn, MA). In fact, many steps of protocols herein may be performed using reagents and material sold under the product name truXTRAC FFPE total NA (tNA) Ultra Kit by Covaris. The FFPE sample may be immersed in the tissue lysis buffer/PK solution mix and sonicated in a ultrasonication instrument according to manufacturer instructions for paraffin emulsification. The steps may be performed in laboratory test tubes, wells of a plate, microcentrifuge tubes, or tubes in a multi-tube strip. The description herein is given in terms of individual microcentrifuge tubes such as the 0.5 mL tube sold as the AFA-TUBE PP Screw-Cap 0.5 mL tube by Covaris. However, one of skill in the art will appreciate that mixtures, emulsification, sonication, centrifuging, column separation, bead clean-up, and other such steps may be performed in tube strips (e.g., a strip of 8 tubes), multi-well plates, traditional (e.g., glass) test tubes, larger (e.g., 50 mL) conical tubes such as those sold under the trademark FALCON by Corning (Corning, NY), or other such containers.

After the tube is collect, it is centrifuged, e.g., spun at 5 k g for about 15 minutes, to form a pellet that includes DNA. The described protocols provide high quality DNA, suitable or sequencing, with high yield from FFPE tissue samples. Preferably, the pellet is rehydrated with a suitable buffer such as buffer BE from Covaris and more preferably a tissue lysis buffer/PK solution mix is used. The tube may be sonicated to resuspend material of the pellet, and optionally treated with RNase. A DNA purification column may be placed into a collection tube.

Sample is transferred into the column and the tube spun. Following DNA purification protocol instructions, the column is washed with buffer(s) such as BW Buffer and B5 Buffer (Covaris). Finally, the column is eluted with an elution buffer, eluting the DNA from the column. The collected (eluted) DNA may be stored at 2° C. for up to 2 days, or at −20° C. for longer term storage. Methods of the disclosure are provided for producing high quality and high yield sequencing libraries from FFPE-extracted DNA.

### Library Preparation

Having extracted DNA from a sample, methods may include library preparation, which generally includes fragmentation, adaptor ligation, and amplification. When the source is a tumor biopsy, nucleic acids in very small quantities, or preserved (e.g., FFPE) sample, extracted DNA may be fragmented via a fragmentation step that is more gentle and less damaging than conventional protocols. Preferably, the eluate that includes the extracted DNA is sheared or fragmented to yield fragments with an average fragment size of at least about 800 base-pairs. Any suitable approach may be used for shearing including enzymatic shearing, nebulization, sonication, Covaris shearing, or others. In some embodiments, it may be preferable to produce fragments that have an average size with a peak approximately within the range of about 500, preferably at least about 600 or 700, and most preferably at least about 800 base pairs (bp) to 1,000 bp. A cocktail of restriction enzymes may be composed that will, on average, cut genomic DNA on about 800 to 1,000 base intervals. Preferred embodiments use a sonicator or adaptive acoustic focusing (AFA) instrument (Covaris). Embodiments may use a Qubit instrument to evaluate quantity and/or a TAPESTATION automatic electrophoresis instrument to evaluate fragment length, using manufacturer's literature for guidelines for the sonication instrument. One approach is to shear a very small sample to the desired optical density to establish the instrument settings to be used for the bulk of the sample. The resultant shearing protocol produces 800 to 1000 base fragments.

The fragments may be repaired enzymatically. Enzymatic repair on such long fragments can correct specific injuries associated with FFPE storage and handling. Preferably the fragments are treated with enzymes such as DNA glycolase, an apurinic/apyrimidinic (AP) endonuclease, DNA polymerase, and/or ligase. DNA Repair Enzymes and Structure-specific Endonucleases are enzymes which cleave DNA at a specific DNA lesion or structure. Those enzymes can be used for repair of DNA sample degradation due to oxidative damage, UV radiation, ionizing radiation, mechanical shearing, formalin fixation (post extraction) or long-term storage. Those enzymes may perform any combination of base excision repair (BER), DNA mismatch repair, nucleotide excision repair, elimination or repair of large DNA secondary structures using T7 Endonuclease I, nick elimination (ligation), and others.

Preferably end repair is performed, which can be understood as a separate step or as included in enzymatic repair. End repair may use reagents such as the SureSelect XT Library Pep Kit ILM from Agilent or the IDT xGen cfDNA & FFPE Library Preparation Kit, performed in a thermocycler, e.g., as described in Agilent, 2021, SureSelectXT Target Enrichment System for the Illumina Platform, Protocol, Manual part number G7530-900000 by Agilent Technologies, Inc. (102 pages), or as described in IDT, 2022, xGen cfDNA & FFPE DNA Library Prep v2 MC by Integrated DNA Technologies (18 pages), both incorporated by reference.

In some embodiments, the end-repaired fragments are purified using magnetic beads and a magnetic separation device. A bead to DNA fragment ratio of about 0.7× may be used. That ratio of beads (e.g., about 45 μL AMPure XP beads to about 100 μL end-repaired DNA sample) is mixed, incubated, and placed on a magnetic stand. Due to ingredients in the bead mixture (e.g.,

PEG) the charged DNA backbone holds DNA to the beads. An important feature of this embodiment of the disclosure is the minimal or low-bead ratio, which, in combination with the fragment length and subsequent steps, provides high quality, high-yield sequencing libraries from FFPE samples. Enzymes or other reagents may be washed away and DNA may be eluted into a ligation mix.

Methods may include ligating adaptors to the fragments to form adaptor-ligated fragments. Any suitable approach may be used. Some embodiments include dA tailing the 3′ end of the fragments (e.g., using a dA-tailing master mix, e.g., from Agilent) and ligating suitable adaptors. Optionally, a bead cleanup step like above may be performed between dA tailing and ligation. Preferred embodiments add paired-end or Illumina Y adaptors. One kit and protocol well suited for use within this protocol is the xGen cfDNA & FFPE DNA Library Prep Kit sold by Integrated DNA Technologies, Inc. (Coralville, IA). The adaptor ligated fragments may be subject to a size-selection step to isolate selected adaptor-ligated fragments with an average size within a range of about 500 to about 1000 base-pairs from unwanted material. More specifically, preferred embodiments use a tight size selection for fragments in the range of about 550 to about 900 bp.

The selected adaptor-ligated fragments may be amplified to obtain amplicons. In most cases, it will be suitable to amplify only a portion of the fragments (the PCR input), and the remainder may be kept in a freezer. The PCR input is combined with PCR reaction mix (primers, buffer, dNTP, polymerase) typically according to instructions from a reagent vendor. E.g., 35 μL PCR reaction mix with 15 μL PCR input. The tube is thermocycled. In most cases, five cycles will produce adequate yield at this stage. The result is a plurality of clonal amplicons copied from nucleic acid in a tumor sample. The amplicons may have sequencing adaptors or any suitable primer binding sites at either or both ends. At this stage, a library preparation is complete.

The described extraction and library preparation protocols are optimized, compared to commercially available kits and protocols, to compensate for damage that is characteristic of FFPE samples and their extraction. For example, after emulsification of the paraffin, DNA may be subject to a limited fragmentation process designed to only fragment the DNA to a large peak length not found in existing protocols. After enzymatic repair, the fragments are subject to a gentle bead cleanup with only a fraction of a quantity of beads found in commercial protocols. The resultant fragments are subject to adaptor ligation and an extra purification with size-selection step is performed on the adaptor-ligated fragments prior to amplification. Each of the steps—limited fragmentation, gentle bead clean-up, and purification after adaptor ligation with size-selection step—may contribute importantly to the preparation of high-quality sequencing libraries from FFPE samples. Compared to prior commercial protocols, other steps may be optimized. For example, after DNA repair and bead clean, high input quantities may be used for adaptor ligation and amplification (e.g., 500 ng instead of 250 ng). In another example, an additional bead clean-up step is added to the protocol after amplification. In another example, the input material may be tested with a quality control assay such as a digital (dPCR) test to qualify the length of fragments. After amplification, another dPCR may be used to quantify yield. In another example, outputs of amplification may be grouped by library yield and groups (based on yield) may be combined for multiplex sequencing. Combining sample first by library yield ensures that sequencing is performed on substantially equimolar library products, which greatly promotes uniform quality of sequencing results.

Because protocols of the invention are useful to prepare high-quality sequencing libraries from FFPE tissue, they are useful for discovering tumor-specific mutations (e.g., structural variants) when applied to FFPE tumor samples, such as from a tumor biopsy. Once a tumor-specific somatic structural variant is known and described, that variant may be used subsequently as a marker for the presence of that tumor. In fact, protocols for library preparation from FFPE tumor samples are designed to yield, and have been found to yield, sequencing libraries of sufficient quality to identify somatic variants even without so-called “matched normal” DNA sequences from the same patient. Instead, tumor DNA may be extracted from an FFPE tumor sample according to protocols described herein, sequenced, and analyzed to identify putative structural variants (SVs). Algorithms are then applied to exclude artifacts of sample-handling and to compare the remaining putative SVs to references and/or databases to filter out germline SVs. Such an analysis may provide an identification of tumor-specific somatic SVs actually present in a patient's tumor DNA. That information is then used to design reagents to assay future samples from the patient for those same tumor-specific somatic SVs. In addition, tumor-specific variants discovered using processes of the invention may be useful as generalized markers for structural variants. For example, an informatics pipeline may be used to design amplification primers and fluorescent probes for the detection of such variants by a digital PCR assay. Particular embodiments identify tumor-specific SVs present in a patient's tumor DNA and then use an informatics pipeline to design primers and fluorescent hydrolysis probes useful for detecting by digital PCR those SVs in cell-free tumor DNA in blood or plasma, e.g., from a liquid biopsy.

### Sequencing

Nucleic acid obtained according to methods of the disclosure is preferably sequenced to obtain sequence data. For example, methods may include sequencing DNA from a tumor sample from the subject to obtain sequence reads.

Sequencing may be by any method known in the art. Suitable DNA sequencing techniques may include the dideoxy chain-termination sequencing technique known in the art as Sanger sequencing, which uses labeled terminators and gel separation in a slab or capillary. Sequencing may include the sequencing by synthesis using reversibly terminated nucleotides and the detection of pyrophosphate in the technique known as pyrosequencing commercialized by ROCHE 454. Sequencing may proceed by techniques that include allele specific hybridization to a library of labeled oligonucleotide probes, sequencing by synthesis using allele specific hybridization to a library of labeled clones that is followed by ligation, real time monitoring of the incorporation of labeled nucleotides during a polymerization step, polony sequencing, and SOLID sequencing. Separated molecules may be sequenced by sequential or single extension reactions using polymerases or ligases as well as by single or sequential differential hybridizations with libraries of probes. Sequencing may be performed using one of the single molecule, long read sequencing platforms commercialized by HELICOS, PACIFIC BIOSCIENCES, or OXFORD NANOPORE.

Sequencing techniques and instruments that may be used include, for example, those offered by ILLUMINA, INC. or ULTIMA GENOMICS. Illumina sequencing is based on the amplification of a sequencing library described above on a solid surface of a flow cell using fold-back PCR and anchored primers. Amplicons of adaptor-ligated fragments that constitute the sequencing library are annealed to oligos attached to the surface of flow cell channels that are extended by which the amplicons are bridge amplified. The fragments become double stranded, and the double stranded molecules are denatured. Multiple cycles of the solid-phase amplification followed by denaturation can create several million clusters of approximately 1,000 copies of single-stranded DNA molecules of the same template in each channel of the flow cell. Primers, DNA polymerase and four fluorophore-labeled, reversibly terminating nucleotides are used to perform sequential sequencing. After nucleotide incorporation, a laser is used to excite the fluorophores, and an image is captured, and the identity of the first base is recorded. The 3′ terminators and fluorophores from each incorporated base are removed and the incorporation, detection and identification steps are repeated. Sequencing according to this technology is described in U.S. Pat. 7,960,120; U.S. Pat. 7,835,871; U.S. Pat. 7,232,656; U.S. Pat. 7,598,035; U.S. Pat. 6,911,345; U.S. Pat. 6,833,246; U.S. Pat. 6,828,100; U.S. Pat. 6,306,597; U.S. Pat. 6,210,891; U.S. Pub. 2011/0009278; U.S. Pub. 2007/0114362; U.S. Pub. 2006/0292611; and U.S. Pub. 2006/0024681, each of which are incorporated by reference in their entirety.

Sequencing generates output and for short-read, ensemble sequencing platforms such as the ILLUMINA platform, the output comprises a large number of short sequencing reads typically accessible from the ILLUMINA system in a computer file format known as FASTQ.

The sequencing instrument and technique relates to the biochemistry of base determination and also implicates read length and read number, with consequences for read assembly. For example, the output from Sanger sequencing on a glass-capillary instrument provided by ABI is typically a small number of medium length (several hundred bases) chromatograms that are provisional “called” (interpreted) as bases by software and presented for human verification. Long read sequencing provide single or low numbers of much longer (>1,000) base reads. Short read sequencing (e.g., ILLUMINA) provides a large number (e.g., millions) of short reads (e.g., 50 or fewer bases) that are typically mapped to a reference and/or assembled de novo to show the original sequence. Illumina is accepted as an industry standard example of a next-generation sequencing (NGS) platform. Whatever instrument or technique is used, methods may include one or any combination of suitable “coverage” strategies, which involve determinations of what targets to sequence and at what coverage.

Coverage strategies may include, for example, transcriptome sequencing in which all RNA transcripts are sequence redundantly, re-sequencing in which a presumptively very similar genome is known, and only highly variable targets are sequenced, whole exome sequencing in which all expressed genes or exons are sequenced, or other coverage strategies. Even with a particular coverage strategy, one may opt for a certain depth of coverage. For example, for some applications, when NGS is used, 30× coverage is considered a standard coverage in which substantially all bases are sequenced redundantly such that each base on average appears in about 30 unique sequence reads. Certain preferred embodiments of the invention use low-pass whole genome sequencing (as used herein, “whole genome sequencing” means that a substantial portion such as at least 80% or 90% of a genome or at least a chromosome is sequenced). Low-pass whole genome sequencing (LP-WGS) is a technique in which each base in the entire genome is sequenced a few times (known as low-depth coverage) e.g., with a depth of coverage below about 5 and as low as 0.1-1 times. By reducing the depth of coverage, the cost of sequencing the whole genome is reduced while maintaining a broad look at the full genome. LP-WGS is described in Christodoulou, 2023, Combined low-pass whole genome and targeted sequencing in liquid biopsies for pediatric solid tumors, NPJ Precision Onc 7:21 and Zheng, 2022, Experience of low-pass whole genome sequencing-based copy number variant analysis, Diagnostics (basel) 12 (5): 1098, both incorporated by reference.

Whatever technique and coverage is employed, methods include sequencing nucleic acid from a tumor. In certain preferred embodiments, LP-WGS is used to sequence substantially at least about 90% of a tumor genome at a coverage of about 5× or lower. The sequencing provides sequence data of the tumor nucleic acids. The sequence data may be analyzed to create a personalized tumor mutation profile, which includes any potential tumor variants and/or mutations.

A variety of different variants and mutations may be tracked using the tumor mutation profile. Typically, these variants are structural variants. Structural variants (SVs) are genomic abnormalities that may amplify, delete, or rearrange genomic regions of a tumor. It is possible and, in fact, common for more than one SV to occur in the same tumor. As used herein, an SV generally refers to a rearrangement, duplication, or deletion of a segment of length of at least about 1,000 bases. Methods of the disclosure may also be used to detect tumor-specific polymorphisms and/or small indels.

### Detection of Tumor-Specific Variants

The disclosure includes methods for analyzing sequence reads, as may be obtained from nucleic acid from tumors, to identify structural variants (SVs), and optionally filter out any putative structural variants that are not somatic (e.g., germline SVs or artifacts from sample processing or sequencing) to identify SVs that may be specific to the tumor, i.e., tumor variants. Methods may include comparing tumor sequence to a reference by one or more algorithms, identifying structural variants in the tumor nucleic acid, and designing primers to specifically amplify those tumor variants. Sequence reads from tumor nucleic acid may first be cleaned up, mapped to a reference, and or subject to computational workflows to detect SVs.

Reads can be cleaned using known software methods such as fastp as described in Chen, et al., 2018, fastp: an ultra-fast all-in-one FASTQ preprocessor, Bioinformatics, 34 (17): 1884-i890, incorporated by reference. Cleaning may include trimming adapter sequences, removing low quality bases at the ends of reads and artifacts such as polyG tails. In some embodiments cleaning may include removing reads shorter than 30 bp instead of a standard 15 bp limit that may inadvertently select out shorter valid sequence reads resulting from sample fixation. Cleaned reads can be subjected to quality control using, for example, the FastQC available from the Babraham Institute, Cambridge UK.

Sequence reads, obtained via any known method, may be mapped to a reference using assembly and alignment techniques known in the art or developed for use in the workflow. Various strategies for the alignment and assembly of sequence reads, including the assembly of sequence reads into contigs, are described in detail in U.S. Pat. 8,209,130, incorporated herein by reference. Sequence assembly can be done by methods known in the art including reference-based assemblies, de novo assemblies, assembly by alignment, or combination methods. Sequence assembly is described in U.S. Pat. 8,165,821; U.S. Pat. 7,809,509; U.S. Pat. 6,223,128; U.S. Pub. 2011/0257889; and U.S. Pub. 2009/0318310, the contents of each of which are hereby incorporated by reference in their entirety. Sequence assembly or mapping may employ assembly steps, alignment steps, or both. Assembly can be implemented, for example, by the program ‘The Short Sequence Assembly by k-mer search and 3′ read Extension’ (SSAKE), from Canada's Michael Smith Genome Sciences Centre (Vancouver, B.C., CA) (see, e.g., Warren et al., 2007, Assembling millions of short DNA sequences using SSAKE, Bioinformatics, 23:500-501, incorporated by reference). SSAKE cycles through a table of reads and searches a prefix tree for the longest possible overlap between any two sequences. SSAKE clusters reads into contigs.

In certain embodiments, reads are aligned to a reference human genome using Burrows-Wheeler Aligner version 0.5.7 for short alignments, and genotype calls are made using Genome Analysis Toolkit. See McKenna et al., 2010, The Genome Analysis Toolkit: a MapReduce framework for analyzing next-generation DNA sequencing data, Genome Res 20 (9): 1297-1303, incorporated by reference (aka the GATK program). Reads may be assembled using SSAKE version 3.7. The resulting contiguous sequences (contigs) can be aligned to the reference (e.g., using BWA). In some embodiments, the reference genome may include GRCh38.

A workflow for SV detection from sequence reads and for primer design may be automated using tools such as Snakemake or Nextflow and custom programming using R or Python, for example, to link input/output across the various workflow steps. Some embodiments employ a computational pipeline that uses two or more different algorithms, each intended for finding SVs, to call putative SVs and merge the results. The computational pipeline may be used for mapping reads to a reference by a first algorithm (in a first mapping) and also by a second algorithm to identify SVs by each algorithm and then selecting the better result or merging the results of the multiple mapping steps to describe the structural variants. One of the algorithms may be a graph-based algorithm. In preferred embodiments, the first algorithm adds the reads to a genomic graph and finds a path through the graph best supported by the reads. This approach may be implemented by a suitable software platform such as the de Bruijn graph-based assembler GRIDSS. Methods may include software, tools, and techniques described in Cameron, 2017, GRIDSS: sensitive and specific genomic rearrangement detection using positional de Bruijn graph assembly, Genome Research 27 (12): 2050-2060 and Cameron, 2021, GRIDSS2:

comprehensive characterization of somatic structural variation using single break-end variants structural variant phasing, Genome Biol 22 (1): 202, both incorporated by reference. In order to adapt to low-pass whole genome sequencing samples, variant calling parameters in the GRIDSS program may be changed including, for example, shortening the minimum length, minimum variant calling score, and minimum variant calling breakpoint quality and increasing the minimum variant calling size.

Preferably, the second algorithm aligns read pairs to a reference and searches for genomic regions in the reference where a significant number of read pairs align to the reference in positions anomalous with an empirical insert size distribution for the read pairs. That algorithm may be implemented by a software platform such as BreakDancer. Methods may include software, tools, and techniques described in Chen, 2009, BreakDancer: an algorithm for high resolution mapping of genomic structural variation, Nat Methods 6 (9): 677-681, incorporated by reference. SplitSeq may be used to refine SV calls made by the first or second algorithm, especially those made with BreakDancer as described in Olsson, et al., 2015, Serial monitoring of circulating tumor DNA in patients with primary breast cancer for detection of occult metastatic disease, EMBO Mol Med, 7 (8): 1034-1047, incorporated herein by reference in its entirety. SplitSeq can be used to reconstruct the exact fusion sequence based on split reads and read pairs with one unmapped mate. Discordant reads can be re-aligned to reduce false positive SV calls. After merging of the SV calling paths using the first and second algorithms, the putative SVs can be annotated with genes that overlap SV breakpoints,

Methods may include filtering SVs that were identified by the mapping workflows to remove germline SVs and/or sample handling artefacts, thereby providing a set of somatic SVs, or tumor variants, present in the tumor DNA. The filtering step may involve comparing the putative SVs to at least one database of known germline SVs and removes matches from the putative SVs. It is understood that some of modern genomics is predicated on a view that there are sequenced and published “reference genomes” and that a sequencing genetic material from a subject gives data that can be analyzed by comparison to the reference. The language of variants sometimes refers to differences between the subject and the reference as a variant in the subject. From that perspective, many people may be born with benign germline SVs (relative to the reference). When sequencing DNA according to the embodiments herein, a variant calling pipeline may find those benign germline variants. Typically, one is more interested in somatic mutations that are specific to a tumor (from which the FFPE sample was created) as those may be used to specifically target and track tumor development, remission, and recurrence. Thus, all SVs found by sequencing are preferably filtered to remove benign germline variants from the putative set, leaving a set of tumor-specific somatic SVs. Filtering may include comparing to a database of known SVs to remove from consideration those that are documented to be benign. Such a databaes may include gnomAD v2.1 SVs available from the Broad Institute, Cambridge, MA; Genome in a Bottle SVs (see Chapman, et al., 2020, A crowdsourced set of curated structural variants for the human genome, PLOS Comp Bio, 16 (6): e1007933, incorporated herein by reference in its entirety); or dbVar v186 SVs available from the National Center for Biotechnology Information.

Those workflows provide for mapping the sequence reads to a reference and identifying read mappings that indicate a structural variant in the tumor nucleic acid, relative to the non-tumor nucleic acid of the subject. That structural variant is tumor specific. It is a variant specific to the tumor, herein referred to as a tumor variant. Using methods of the disclosure, the tumor variant is found by sequencing tumor nucleic acid and analyzing the sequence data. A feature of the disclosure is that such a tumor variant is confirmed by orthogonal testing. Thus, the invention provides methods for analyzing tumor nucleic acid from a tumor from a subject to discover one or more variants that are specific to the tumor and confirming by orthogonal testing that nucleic acid of the tumor harbors the variants and that the variants are specific to the tumor and thus useful as a tumor biomarker in an independent assay for the presence of the tumor in the subject.

### Orthogonal Validation

The disclosed methods are used to detect and report one or more tumor-specific variants that constitute a tumor signature. Following identification of the tumor-specific variants, the disclosure includes performing additional analysis to validate that any such variant is, in fact, present in the tumor genome (to remove false positives) and also, in fact, not present in healthy, non-tumor DNA (correct for false negatives).

Preferably, the method used for confirmatory testing of a tumor variant in a patient is orthogonal to the method used to create the tumor mutation profile, or signature. The use of this orthogonal testing method offers a distinct error profile from the method to identify the candidate SVs. That is, where a tumor variant is detected by NGS techniques, the variant is validated by some separate, other technique. While either technique may by itself have some bias or error, the use of an orthogonal technique confirms the variant but only with different, dissimilar potential sources of bias or error. There is a very low probability that an error introduced by a primary detection technique will be exactly mimicked by an orthogonal detection technique. Thus, consensus between the two detection techniques is strong indication that the detected tumor variant is a true tumor variant. Use of an orthogonal testing method mitigates the risk of false positives and/or negatives. In certain embodiments, the orthogonal testing method comprises testing the detected tumor variant that was sequenced against normal tissue to test for presence of clonal hematopoiesis of indeterminate potential (CHIP) or Germline SVs. The orthogonal testing method may also be performed against an unmatched control (i.e., DNA not derived from the patient) to detect the presence of spurious amplification or false positives. The orthogonal testing method can test for the presence of clonal hematopoiesis of indeterminate potential (CHIP) or Germline SVs as well as for SVs present in the tumor. The orthogonal testing method may also be used to accurately determine specific characteristics of a variant, e.g., the copy number of a SV.

The orthogonal test may be performed by a variety of different methods. Various testing methods may be chosen depending on the specific needs of the patient, the availability of the test to the physician, or various other factors. Any orthogonal test may be used as long as it carries a separate error profile to the initial methods of the previous step. Preferred embodiments use NGS as a primary variant detection technique to detect a variant and use a suitable orthogonal detection technique to confirm the detected variant.

Any suitable test may be used for the orthogonal detection technique including, for example, quantitative PCR (qPCR), ELISA, restriction fragment length polymorphism (RFLP) and similar, optical mapping, reverse transcription polymerase chain reaction (RT-PCR), transcription-mediated amplification (TMA), ligase chain reaction (LCR), targeted re-sequencing, RNA sequencing, Sanger sequencing, single molecule sequencing, atomic force microscopy sequencing, fluorescent in situ hybridization (FISH), a fluorescent-probe based DNA microarray, in vitro transcription, nanopore sequencing, long read sequencing and protein sequencing or detection, others, or combinations thereof.

In one preferred embodiment, the orthogonal detection technique includes digital PCR (dPCR). Having detected tumor SVs by the preferred detection technique such as NGS as described, primer pairs are designed that would specifically generate amplicons in the presence of the tumor SVs but would not generate any amplicon when exposed to only health, non-tumor nucleic acid. One preferred strategy is to, for each SV, design a primer pair that flanks a breakpoint of the SV. Then, a sample comprising tumor nucleic acid (e.g., a biopsy) is obtained and partitioned into aqueous partitions with the primers, PCR reagents, and fluorescent probes specific for amplicons generated when the primers amplify from the SV. The sample is preferably diluted for partitioning such that, on average, each partitions receives zero or one molecule of tumor nucleic acid. Such digital PCR setups are known in the art and it us understood that a trivial number of partitions will receive greater than 1 template molecule without biasing the results. The partitions may be aqueous droplets or wells in a plate. The partitions are thermocycled and each partition is read for fluorescent signal from the probes. The number of positive partitions corresponds to the number of targets in the sample. A sample comprising only non-tumor DNA may be run as a negative control to confirm that the primer pair is specific for a tumor SV. The results of that orthogonal dPCR assay thus may be used to validate that the SV detected by the primary, NGS detection technique is a true tumor-specific variant.

In some embodiments, the orthogonal detection technique includes Sanger sequencing. Sanger sequencing does not require the same combination of PCR and bridge amplification steps that are involved in NGS and the output of Sanger sequencing is chromatograms that are much longer than short-reads e.g., from NGS. Due to the output length, Sanger sequencing is much less prone to assembly errors for short copy number variations such as dinucleotide and trinucleotide repeats and also less prone to base calling errors by software in the presence of small indels. Sanger sequencing instruments have matured over decades of use and refinement and use electrophoretic gel separation in glass capillaries of fluorescently labeled fragments. Sanger sequencing is good enough that it is accepted in the primary literature as the “gold standard” against which any new sequence analysis technique must be proven. There is one known issue of a mis-labeled fragment being driven through the capillary past the detector simultaneously with correctly-labeled fragments. But that issue is complementary to the types of problems that NGS has with amplification bias and homopolymer runs. For those reasons, Sanger sequencing offers an excellent orthogonal validation technique for use in methods of the disclosure.

In certain embodiments, the orthogonal detection technique includes atomic force microscopy (AFM). AFM uses a cantilever tip that can interact with DNA where input force required for motion of a base is characteristic allowing base sequence to be read from the instrument run. AFM is sensitive to secondary structures, bound proteins, and packaging. AFM is potentially useful to scan DNA in situ, e.g., within a fixed sample without liberation into solution. As tumor variants will disrupt assembly of the transcription complex and knock out binding sites for DNA binding proteins, AFM can show the bound proteins—or lack of bound proteins—and absence of complexes and structure that would only be consistent with the tumor variants identified by NGS.

The orthogonal detection may optionally includes restriction fragment length polymorphism (RFLP) and similar techniques (such as amplified fragment length polymorphism (AFLP) and/or optical mapping. Techniques such as RFLP, AFLP, and optical mapping all give a characteristic and very specific output based on where, or in what pattern, restriction enzyme cut DNA. A tumor SV will change a restriction pattern, relative to non-tumor DNA, in two significant ways. As “SV” stands for structural variant, the tumor DNA differs from non-tumor DNA because large (>1 kb) segments have been moved, removed, duplicated, or inverted. This will create a very different restriction pattern. Additionally, tumors are hyperproliferative and tend to have extensive hypomethylation of genes and promoters where, for example, methylation of promoter of an oncogene in healthy, non-tumor DNA inhibits expression of the oncogene, a common phenomenon in cancer is hypomethylation and associated expression of the oncogene. Classes of restriction enzymes are methylation specific and such methylation-specific restriction enzymes may be used once a tumor SV is identified by NGS to validate that restriction patterns that would be expected in the tumor DNA are observed in the tumor DNA.

In some embodiments, the orthogonal detection technique includes FISH or a fluorescent microarray. Both are examples of techniques in which sample DNA is exposed to sequence-specific probes that anneal to the sample DNA when and only when a target of interest is present. Once a tumor SV is identified by NGS, a plurality of probes (e.g., dozens, hundreds, or thousands, etc.) in which some number can be designed to hybridize only to nucleic acid that includes the tumor SV; some hybridize only to nucleic acid that does NOT include the tumor SV, some hybridize to both tumor and non-tumor nucleic acid, and some may hybridize only to some extrinsic control nucleic acid. Tumor nucleic acid and non-tumor nucleic acid are exposed to the probe set and patterns of hybridization are read (e.g., fluorescently) to validate that a pattern that would be expected (if the SV is a true tumor-specific SV) is observed in the assay data. FISH may be preferred where the original sample is, for example, an FFPE slice of the tumor on a slide, as the FISH probes may be used to interrogate the slice in situ on the slide. A microarray may be preferred for a wet tumor biopsy such as a fine needle aspirate as material from the subject can be (optionally disaggregated, e.g., using a proteinase in a sample tube) washed over the microarray.

Certain embodiments of the invention use protein expression. Noting that genomes include genes that code for proteins, one consequence of the central dogma of molecular biology is that an SV in a gene will likely change the associated protein product. Once a tumor SV has been identified by NGS, that variant identification may be validated by transcription and/or translation of the tumor nucleic acid and analysis of the resultant protein product to validate that the observed protein product is what would be expected, given the identified tumor SV. For example, tumor DNA may be subject to in vitro transcription (IVT) and translation into protein. Or, the tumor DNA may be cloned into a host (e.g., E. coli) and expressed as protein. The protein may then be analyzed. For example, a protein binding technique such as an enzyme-linked immunosorbent assay (ELISA) may be used to confirm that expected proteins are present. In another example, the protein may be analyzed by mass spectrometry, e.g., time-of-flight MS, to determine if it fragments into fragments with expected mass/charge ratios. Any other suitable method of protein analysis or validation of a detected tumor variant is within the scope of the disclosure and may be used.

Because the detected variant is validated by an orthogonal detection technique that is unlike the primary detection technique, any bias or systematic error in either technique is corrected for by the inclusion of the other. Where NGS reflects bias from amplification, systemic error associated with short-read assembly with repeats, or struggles with homopolymer run, the orthogonal technique may be true single molecule sequencing technique (Oxford, PacBio), a physical analysis (AFM), an art-recognized gold-standard sequencing technique (Sanger), a digital target technique (dPCR), a protein analysis technique (IVT, cloning, ELISA, ToF-MS), a fluorescent probe technique (FISH, DNA microarray), or any other suitable technique. Because the identified tumor SV is validated by the orthogonal technique, the SV is shown to be a true and reliable tumor-specific variant, the presence of which is truly indicative of the presence of the tumor in a sample. Embodiments of the disclosure provide improved methods for the detection of tumor variants indicative of MRD. These methods utilize sequencing of tumor nucleic acid and an orthogonal validation method to accurately detect and verify the presence of a tumor variant in a patient. Methods of the present disclosure correct for bias or systemic error in analytical methodologies.

Having validated the identity of the truly tumor-specific variant, the tumor SV may be used in an assay for the presence of the tumor, particularly in an assay for minimal residual disease after treatment for the tumor. In particular, a primer pair may be designed that amplifies the SV or that flanks a breakpoint of the SV and amplifies a segment containing the breakpoint. When a patient is treated to eradicate the tumor, the primer pair can subsequently be used to detect whether any tumor nucleic acid is still present in a sample from the patient.

The primer pair may have particular utility in an assay for minimal residual disease (MRD) and in particular an MRD assay that involves liquid biopsy and digital PCR to detect cDNA.

Blood may be drawn from the patient and sent to the laboratory performing the test. That laboratory may obtain the blood or plasma sample (e.g., in a blood collection tube or similar container), and prepare a digital PCR reaction. The sample may be partitioned into aqueous partitions along with PCR reagents, the primer pair (several primer pairs where the assay is for a tumor signature that includes several tumor SVs identified by detection method with orthogonal validation according to methods of the disclosure), and fluorescent probes specific for amplification products of the primer pair(s). The partitions may be subject to thermocycling and fluorescent signals may be read from the partitions using, e.g., a fluorescence detector. A number of the partitions that give a fluorescent signal may be taken as a quantitative measure of an amount of tumor nucleic acid in the blood draw. The presence of the tumor nucleic acid in the sample may be reported as evidence of minimal residual disease after the treatment to eradicate the tumor.

In certain embodiments, a list of detected SVs are ranked. The top ranked SVs may be selected for orthogonal validation. Depending on the results of the orthogonal validation, subsequent SVs could further be validated and/or sub-populations of the orthogonally validated SVs may be used for patient testing.

As used herein, where the variant is identified or detected with a primary detection technique and an orthogonal detection technique, orthogonal may be understood to mean that one of the techniques may be susceptible to a bias, challenge, difficulty, or source of systematic error (collectively, “error”) that is recognized in the art and that the other technique is recognized in the art as not being susceptible to the same error. Examples of error include amplification biases in sample preparation and variant detection workflows that rely on polymerase chain reaction. In another example, sequencing-by-synthesis may have difficulty sequencing long homopolymer runs. In another example, read assembly from short read sequencers may be difficult for genomic regions with duplications or high copy number amplification. In methods herein, a selected orthogonal detection technique employs principles of physics, biochemistry, molecular biology, and/or bioinformatics that are not susceptible to the same source of error as the primary detection technique. Thus it may be understood that methods of the invention may include analyzing sequence data (from nucleic acid from a tumor), using a first detection method, to identify a tumor-specific variant and analyzing the nucleic acids using an orthogonal method, wherein the first method is associated with a first error and the orthogonal method is not associated with the first error. The potential association with error will be understood to include those issues that are art-recognized. For example, primary, peer-reviewed literature reports that Sanger chromatograms sometimes exhibit two colors at a single peak location. That ambiguity is art-recognized and is an error (as used herein) associated with Sanger sequencing. In another example, the literature reports that assembly of short reads from NGS instruments (“short read assembly”) is error-prone for regions of genomic duplication. That error is associated with NGS instruments such as the ILLUMINA platform but is not associated with Sanger sequencing.

Methods of the invention may use an orthogonal detection technique that is two-way orthogonal to the first detection technique in that each method is associated with an error that the other method is not associated with (first method is associated with a first error, orthogonal method is associated with a second error, first method is not associated with the second error, orthogonal method is not associated with the first error). In methods of the invention, the first variant detection method or technique and the orthogonal variant detection method or technique are used to detect a variant in nucleic acid from a tumor, i.e., tumor nucleic acid.

### INCORPORATION BY REFERENCE

References and citations to other documents, such as patents, patent applications, patent publications, journals, books, papers, web contents, publicly accessible databases, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A method for the detection of a tumor variant comprising:
sequencing nucleic acids from a tumor from a subject to generate sequence data;
analyzing the sequence data to identify a tumor-specific variant; and
analyzing the nucleic acids using an orthogonal method to validate the presence of the tumor specific variant in the nucleic acids from the tumor.

2. The method of claim 1, wherein the tumor variant is a structural variant.

3. The method of claim 1, wherein the tumor variant is a single nucleotide polymorphism (SNP), an indel (insertion-deletion), a single nucleotide variant (SNV), a deletion, a rearrangement, or an amplification.

4. The method of claim 1, wherein the SNV is selected from a somatic variant or variant for patient identification.

5. The method of claim 1, wherein the tumor variant involves rearrangement, duplication, or deletion of a genome segment greater than 50 bp in length.

6. The method of claim 1, further comprising obtaining a second sample from the subject that is not derived from the tumor.

7. The method of claim 6, wherein the first and second tumor samples are derived from the same sample.

8. The method of claim 1, wherein the nucleic acid is obtained from a formalin-fixed, paraffin embedded slice from the tumor.

9. The method of claim 1, wherein the sequencing method is selected from whole genome sequencing and whole exome sequencing.

10. The method of claim 1, wherein the orthogonal testing method comprises digital PCR (dPCR), ELISA, a restriction digest, or single molecule sequencing.

11. The method of claim 1, further comprising additional testing of the patient and tumor samples to confirm the nature of a tumor variant.

12. The method of claim 1, wherein the detection of one or more tumor variant(s) in tumor tissue but not in control samples constitutes a positive test.

13. The method of claim 1, further comprising biasing the orthogonal method by removing or not analyzing specific primers or variants based on results obtained from previous testing and/or sequencing.

14. The method of claim 1, further comprising providing a primer pair designed to specifically amplify the tumor specific variant and to not generate any amplification product when thermocycled with non-tumor nucleic from the subject.

15. The method of claim 14, further comprising testing a sample from the patient for the presence of the tumor by performing an assay that includes an amplification reaction using the primer pair.

16. The method of claim 1, wherein the assay is performed using a liquid biopsy sample from the patient and the amplification reaction is performed on plasma from the sample to test for the tumor specific variant among circulating tumor DNA (ctDNA) in the plasma.

17. The method of claim 15, wherein the patient has undergone treatment eradicate the tumor and the testing is for minimal residual disease.

18. The method of claim 1, wherein the sample is selected from the group consisting of blood, saliva, plasma, urine, CSF, stool, tumor biopsy, and lymphatic fluid.
